(19) United States
(12) Patent Application Publication
(10) Pub. No.: US 2026/0199145 A1
(43) Pub. Date: Jul. 16, 2026

(54) REMOVAL DEVICE FOR REMOVING AN OBJECT FROM A CAVITY AND METHOD

(71) Applicant: Yuliya BORUCH, Jamaica Estates, NY (US)

(72) Inventor: Yuliya BORUCH, Jamaica Estates, NY (US)

(21) Appl. No.: 19/136,070

(22) PCT Filed: Dec. 8, 2023
(86) PCT No.: PCT/US2023/083078

§ 371 Date: Jun. 5, 2025

Related U.S. Application Data
(60) Provisional application No. 63/431,508, filed on Dec. 9, 2022.

(51) Int. Cl. A61F 13/34

(52) U.S. Cl. CPC A61F 13/34

(57) ABSTRACT

A removal device for removing an object from a cavity, such as a retained tampon from a vaginal canal. The removal device has a finger cover, wherein the finger cover has an open end, a closed end, and a cylindrical portion, wherein the finger cover has a plurality of protrusions on at least a portion of an exterior surface of the cylindrical portion, and wherein the finger cover has a grip extending from the closed end. The removal device can also be used in a method for removing retained tampons that involves inserting one or more removal devices into a vaginal cavity, securing the retained tampon with the removal device, and removing the retained tampon with the removal device.

### TECHNICAL FIELD

The present application relates to a removal device for removing an object from a cavity and an associated method. In particular, the removal device described herein can be used to remove an object, such as a retained tampon, from a vaginal canal.

### BACKGROUND

Tampons are feminine hygiene products that can be used to absorb menstrual fluid (e.g., vaginal secretions and menstrual blood) during menstruation. Typically, tampons are made of synthetic and/or organic fibers that are capable of absorbing menstrual fluid. A tampon is generally inserted into the vaginal canal with the assistance of a plastic applicator but can also be manually inserted with the user's hands. After insertion, the tampon absorbs the menstrual fluid and expands within the vaginal canal to reduce and/or prevent leakage of the menstrual fluid.

To remove the tampon from the vaginal canal, a removal string is generally provided and is attached to the tampon. The user can pull on the provided removal string to assist with the removal of the tampon from the vaginal canal. The tampon, however, can become stuck within the vaginal canal for a variety of reasons making its removal difficult. For example, the removal string can become detached from the tampon, the removal string can become inserted into the vaginal canal, the user can forget to remove the tampon for an extended period of time, the user can engage in sexual activity that pushes the tampon deeper into the vaginal canal, etc. Such stuck tampons can also be referred to as “retained tampons.”

Retained tampons should be timely removed from the vaginal canal to avoid unwanted consequences, such as menstrual toxic shock syndrome and other infections. The removal of retained tampons from the vaginal canal, however, can be difficult, especially for the user to do by themselves. As a result, users commonly seek assistance from medical professionals to remove retained tampons. Medical professionals may attempt to remove retained tampons manually (e.g., with their hands), but also can use a vaginal speculum to improve access to the vaginal canal and remove the retained tampons with tools such as forceps. Even though these procedures are effective, the tampon user must travel to the medical office for the procedure, pay fees for the medical procedure, be subject to an invasive procedure, etc. Thus, the need exists for a convenient and effective alternative that can be used by individuals themselves.

Others have attempted to provide alternatives for tampon removal devices. For instance, U.S. Pub. No. 2017/0224557 provides a glove with a tampon pocket that prevents the user's hand and clothing from being contaminated with menstrual fluid. Such a glove, however, does not improve the effectiveness of the tampon removal, but merely makes the removal more sanitary. U.S. Pat. No. 5,740,554 similarly provides a mitten for use by an individual to remove a tampon in a sanitary manner. U.S. Pat. No. 6,687,911 also disclosed a handling aid for a tampon for feminine hygiene having at least two separate finger coverings. Such mittens and handling aids also merely make the removal more sanitary but do not improve the effectiveness of the removal. Thus, the need still exists for tampon removal devices that allow an individual to remove a retained tampon effectively.

In an effort to improve upon and solve the problems of known removal devices, the inventor has discovered the removal devices disclosed herein. The removal devices provided herein provide a safe and effective tool for users to remove retained tampons themselves. Also, the removal devices provide medical professionals with an additional safe and effective tool for the removal of retained tampons. Even though the removal devices disclosed herein were discovered in an effort to provide an improved removal device for retained tampons, the removal devices disclosed herein are not limited to the removal of retained tampons. For example, the removal devices disclosed herein can remove other objects from the vaginal canal. The removal devices disclosed herein can also be used to remove objects from other bodily cavities, such as the mouth, ears, anal canal, rectum, etc. The removal devices disclosed herein can even further be used in a non-medical setting, such as being used in a construction setting (e.g., to remove small pieces from a pipe). Thus, the removal devices disclosed herein are not limited to medical uses.

### BRIEF SUMMARY

In view of the above-mentioned exemplary problems with conventional and known tampon removal devices, the present application provides new and improved tampon removal devices and associated methods.

An embodiment of the present application includes a removal device for removing an object from a cavity comprising a finger cover (1), wherein the finger cover (1) comprises an open end (2), a closed end (3), and a cylindrical portion (4), wherein the finger cover (1) comprises a plurality of protrusions (5) on at least a portion of an exterior surface of the cylindrical portion (4), and wherein the finger cover (1) comprises a grip (6) extending from the closed end (3).

In another embodiment of any of the above-mentioned embodiments, the open end (2) is configured to accept a finger of a user.

In another embodiment of any of the above-mentioned embodiments, the closed end (3) has a convex shape.

In another embodiment of any of the above-mentioned embodiments, the cylindrical portion (4) is right cylinder.

In another embodiment of any of the above-mentioned embodiments, the plurality of protrusions (5) extend radially from the exterior surface of the cylindrical portion (4).

In another embodiment of any of the above-mentioned embodiments, the plurality of protrusions (5) are present on a predefined arc length of the cylindrical portion (4).

In another embodiment of any of the above-mentioned embodiments, the predefined arc length is 20% to 40% of a circumference of the cylindrical portion (4).

In another embodiment of any of the above-mentioned embodiments, the plurality of protrusions (5) are present on the cylindrical portion (4) along a height (h) extending from the open end (2) to the closed end (3).

In another embodiment of any of the above-mentioned embodiments, the plurality of protrusions (5) are evenly spaced on the exterior surface of the cylindrical portion (4).

In another embodiment of any of the above-mentioned embodiments, the plurality of protrusions (5) comprises protrusions having a first height (ph1), protrusions having a second height (ph2), and protrusions having a third height (ph3).

In another embodiment of any of the above-mentioned embodiments, the first height (ph1) is less than the second height (ph2), and wherein the second height (ph2) is less than the third height (ph3).

In another embodiment of any of the above-mentioned embodiments, the protrusions having a first height (ph1) are closest to the open end (2), the protrusions having a third height (ph3) are closest to the closed end (3), and the protrusions having a second height (ph2) are between the protrusions having a first height (ph1) and the protrusions having a third height (ph3).

In another embodiment of any of the above-mentioned embodiments, the closed end (3) has a convex shape having a plurality of protrusions on at least a portion of an exterior surface of the closed end (3).

In another embodiment of any of the above-mentioned embodiments, the grip (6) comprises an extended portion (7) and an enlarged portion (8).

In another embodiment of any of the above-mentioned embodiments, the extended portion (7) is straight.

In another embodiment of any of the above-mentioned embodiments, the extended portion (7) curves toward a center of the plurality of protrusions (5).

In another embodiment of any of the above-mentioned embodiments, the removal device is made from medical-grade silicone rubber.

In another embodiment of any of the above-mentioned embodiments, the object is a retained tampon, and the cavity is a vaginal cavity.

Another embodiment of the present application includes a method of removing a retained tampon comprising inserting the removal device into a vaginal cavity having the retained tampon therein, securing the retained tampon with the removal device, and removing the retained tampon with the removal device.

Other objects, features, and advantages of the present invention will be apparent to those of ordinary skill in the art in view of the following detailed description of the invention and accompanying drawings.

### BRIEF DESCRIPTION OF THE DRAWINGS

The drawings as provided for herein set forth some exemplary embodiments of the removal devices of the present application, the detailed description of which follows. The drawings are merely exemplary and are not intended to limit the invention.

FIG. 1 shows a perspective view of an embodiment of the removal devices disclosed herein.

FIG. 2 shows a side view of an embodiment of the removal devices disclosed herein.

FIG. 3 shows the external surface area of the cylindrical portion of an embodiment of the removal devices disclosed herein.

FIG. 4 shows a picture of an embodiment of the removal devices disclosed herein.

FIG. 5 shows a side schematic view of an embodiment of the removal devices disclosed herein with particular Shore hardness values.

FIG. 6 shows a picture of an embodiment of the removal devices disclosed herein.

### DETAILED DESCRIPTION

The removal devices of the present application are now described by reference to the embodiments. The description provided herein is not intended to limit the scope of the claims, but to exemplify the variety encompassed by the present application. The embodiments are described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

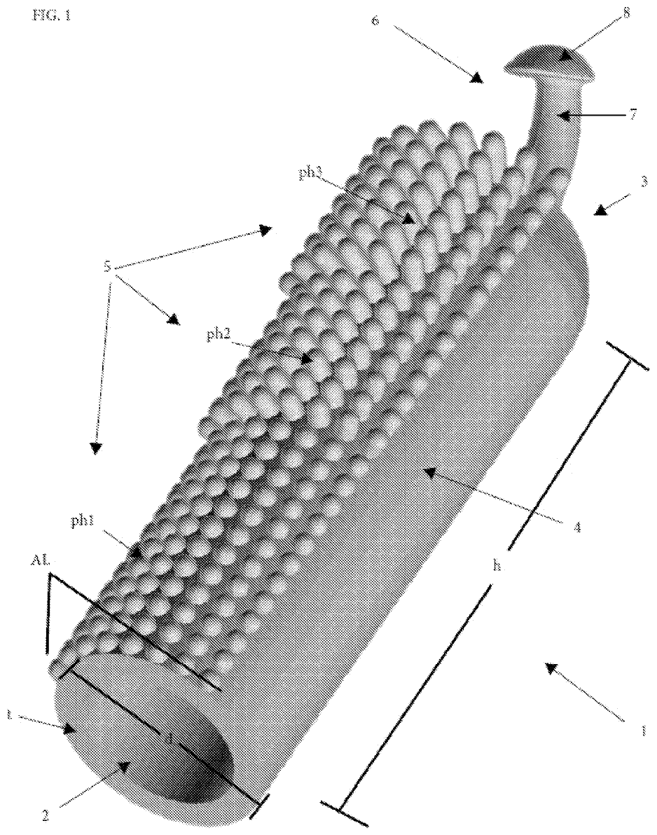

FIG. 1 shows an embodiment of the removal device for removing an object from a cavity. As seen in FIG. 1, the removal device includes a finger cover (1). The finger cover (1) has an open end (2), a closed end (3), and a cylindrical portion (4). The open end (2) is configured to accept a finger of the user. Even though the removal device is generally used on the index finger of the user, the open end (2) can be configured to accept any finger of the user, including the index finger, the middle finger, the ring finger, the small finger (i.e., pinkie finger), and the thumb. The open end (2) can also be configured to accept a tool, rod, extension piece, etc.

The closed end (3) is opposite to the open end (2). The shape of the closed end (3) is not limited. For example, the closed end (3) can be flat, concave, convex, or mixtures thereof. In certain embodiments, the closed end (3) has a convex shape such the convex shape extends away from the open end (2). The convex shape at the closed end (3) of the finger cover (1) may allow for easier and less painful insertion into the vaginal canal because the convex shape can be smooth and not have any edges or corners that could cause irritation to the walls of the vaginal canal.

The finger cover (1) also includes a cylindrical portion (4). The cylindrical portion (4) is present between the open end (2) and the closed end (3). The cylindrical portion (4) can have a height (h) and a diameter (d). The height (h) refers to the distance between the open end (2) and the beginning of the closed end (3). The diameter (d) refers to the outside diameter of the cylindrical portion (4), but the cylindrical portion (4) also has an inside diameter. The cylindrical portion (4) also has a thickness (t), which refers to the thickness of the walls of the cylindrical portion (4). The thickness (t) can be uniform or non-uniform throughout the cylindrical portion (4). For example, the thickness (t) can be a gradient such that the thickness at the open (2) is greater than the thickness at the closed end (3), or vice versa. The thickness (t) can also vary throughout the cylindrical portion (4) such that, for example, the thickness in the middle of the cylindrical portion (4) is less than the thicknesses at either the open end (2) or the closed end (3). The thickness (t) can also be varied to provide grooves or weakened portions that allow the finger cover (1) to bend easily with the joints of the finger. For example, a first groove could correspond to the distal interphalangeal joint (DIP), a second groove could correspond to the proximal interphalangeal joint (PIP), and a third groove could correspond to the metacarpophalangeal joint (MCP). The location of the grooves could vary depending upon the finger that will be inserted into the cylindrical portion (4). Similarly, the height (h) and the diameter (d) of the cylindrical portion (4) can be varied to accommodate different fingers or tools inserted therein. In certain embodiments, the height (h) is 2 inches. In other embodiments, the height (h) can be 1.25, 1.5, 1.75, 2.25, 2.5, 2.75, 3, 3.5, 4 inches, or any range thereof. In certain embodiments, the diameter (d) is 0.6 inches. In other embodiments, the diameter (d) can be 0.1, 0.2, 0.3, 0.4, 0.5, 0.7, 0.8, 0.9, 1 inch, or any range thereof.

The cylindrical portion (4) is generally cylindrical but is not required to have a right cylinder shape. The cylindrical portion (4), instead, can have a right cylinder shape, an oblique cylinder shape, an elliptic cylinder shape, etc. The cylindrical portion (4) can also be tapered such that the diameter of the closed end (3) differs from the diameter of the open end (2). For instance, the diameter of the closed end (3) can be smaller than the diameter of the open end (2) to allow for easier insertion into an orifice, or vice versa.

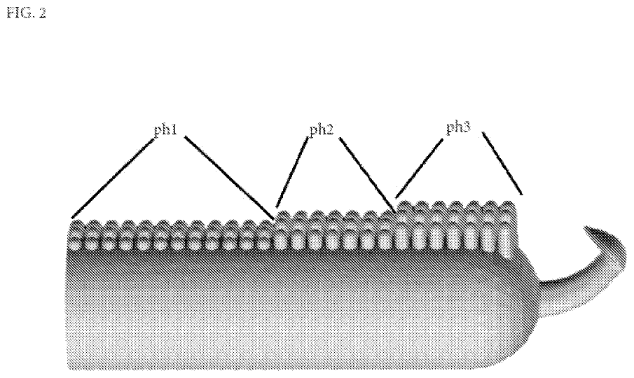

The finger cover (1) has a plurality of protrusions (5) on at least a portion of an exterior surface of the cylindrical portion (4). The plurality of protrusions (5) can, for example, assist in the removal of a retained tampon by embedding the protrusions into the surface of the retained tampon thereby improving the force that can be applied to remove the retained tampon. The plurality of protrusions (5) can cover the entire exterior surface of the cylindrical portion (4) in certain embodiments. In other embodiments, the plurality of protrusions (5) cover only a portion of the exterior surface of the cylindrical portion (4). The plurality of protrusions (5) can be evenly spaced or distributed in a pattern. FIG. 1 and FIG. 2 show the plurality of protrusions (5) being evenly spaced. The plurality of protrusions (5) can extend radially from the exterior surface of the cylindrical portion (4). For instance, when the cylindrical portion (4) has a right cylinder shape, the plurality of protrusions (5) can extend radially from the axis of the cylindrical portion (4). As seen in FIG. 2, the closed end (3) can have a convex shape having a plurality of protrusions on at least a portion of an exterior surface of the closed end (3). The plurality of protrusions on the closed end (3) can extend radially from the axis of the cylindrical portion (4) such that the plurality of protrusions on the closed end (3) continue the evenly spaced pattern of the plurality of protrusions (5) on the exterior surface of the cylindrical portion (4).

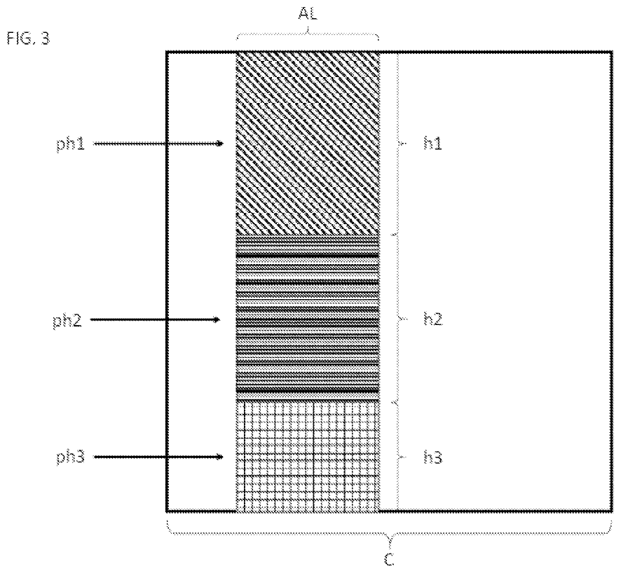

As shown in FIG. 1 and FIG. 3, the plurality of protrusions (5) can cover a predefined arc length (AL) of the cylindrical portion (4). The arc length (AL) can be consistent throughout the height (h) of the cylindrical portion (4) such that the arc length (AL) is the same at the open end (2) and the closed end (3). FIG. 1 and FIG. 3 depict an arc length (AL) that is the same at the open end (2) and the closed end (3). The arc length (AL) can also be different at the open end (2) and the closed end (3) or varied through the height (h) of the cylindrical portion (4). The plurality of protrusions (5) can be present on the cylindrical portion (4) along a height (h) extending from the open end (2) to the closed end (3) or only a portion of a height (h) extending from the open end (2) to the closed end (3). The arc length can be predetermined set such that the plurality of protrusions (5) covers an exterior surface of the cylindrical portion (4) corresponding to the palmar surface of a finger inserted into the cylindrical portion (4). In certain embodiments, the plurality of protrusions (5) covers 10% to 50% of the exterior surface of the cylindrical portion (4). In other embodiments, the plurality of protrusions (5) covers 20% to 40% of the exterior surface of the cylindrical portion (4). In other embodiments, the plurality of protrusions (5) covers 25% to 35% of the exterior surface of the cylindrical portion (4).

The plurality of protrusions (5) can have the same protrusion height or varied protrusion heights. FIG. 2 shows an embodiment having varied protrusion heights. In FIG. 2, the plurality of protrusions (5) includes protrusions having a first height (ph1), protrusions having a second height (ph2), and protrusions having a third height (ph3). The protrusions having a first height (ph1) are closest to the open end (2) and have the shortest protrusion height. The protrusions having a third height (ph3) are closest to the closed end (3) and have the tallest protrusion height. The protrusions having a second height (ph2) are between the protrusions having a first height (ph1) and the protrusions having a third height (ph3) and have a protrusion height between the protrusion height of the protrusions having a first height (ph1) and the protrusions having a third height (ph3). In certain embodiments, the protrusions having a first height (ph1) have a protrusion height of 0.05 inches. In other embodiments, the protrusions having a first height (ph1) have a protrusion height of 0.01, 0.02, 0.03, 0.04, 0.06, 0.07, 0.08, 0.09 inches, or any range thereof. In certain embodiments, the protrusions having a second height (ph2) have a protrusion height of 0.1 inches. In other embodiments, the protrusions having a second height (ph2) have a protrusion height of 0.08, 0.09, 0.11, 0.12, 0.13, 0.14 inches, or any range thereof. In certain embodiments, the protrusions having a third height (ph3) have a protrusion height of 0.13 inches. In other embodiments, the protrusions having a third height (ph3) have a protrusion height of 0.11, 0.12, 0.14, 0.15, 0.16, 0.17, 0.18, 0.19, 0.2 inches or any range thereof.

FIG. 3 shows a flattened exterior surface area of an embodiment the cylindrical portion (4) having protrusions having a first height (ph1), protrusions having a second height (ph2), and protrusions having a third height (ph3). The circumference (C) of the cylindrical portion (4) is depicted to show the relationship to the cylindrical portion (4). As seen in FIG. 3, the protrusions having a first height (ph1) have a height (h1) that is a portion of the height (h) of the cylindrical portion (4). The protrusions having a second height (ph2) have a height (h2) that is a portion of the height (h) of the cylindrical portion (4). The protrusions having a third height (ph3) have a height (h3) that is a portion of the height (h) of the cylindrical portion (4). The heights (h1, h2, and h3) can be even distributed or varied. As shown in FIG. 3, h1 is greater than h2, and h2 is greater than h3. In certain embodiments, h1 can be 30% to 60% of the height (h) of the cylindrical portion (4). In other embodiments, h1 can be 40% to 50% of the height (h) of the cylindrical portion (4). In certain embodiments, h2 can be 20% to 40% of the height (h) of the cylindrical portion (4). In other embodiments, h2 can be 25% to 30% of the height (h) of the cylindrical portion (4). In certain embodiments, h3 can be 20% to 40% of the height (h) of the cylindrical portion (4). In other embodiments, h3 can be 25% to 30% of the height (h) of the cylindrical portion (4).

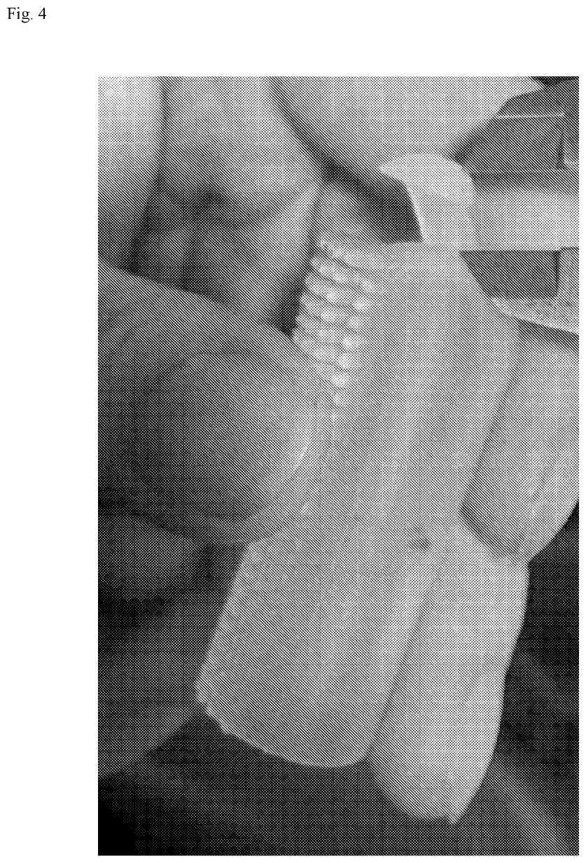

The finger cover (1) has a grip (6) extending from the closed end (3). The grip (6) can assist with, for example, the removal of a retained tampon by hooking into the retained tampon and providing additional removal force. The grip (6) can also extend the length of the user's finger to assist with the removal of retained tampons that become lodged deep within the vaginal canal. The grip (6) can have an extended portion (7) and an enlarged portion (8). The extended portion (7) can be straight or curved. FIG. 1 shows an embodiment having a curved extended portion (7). FIG. 4 shows an embodiment having a straight extended portion (7). The extended portion (7) can extend from the closed end (3) at an angle in the range of 30° to 60° or even from 40° to 90°. In other embodiments, the extended portion (7) can extend at an angle in the range of 40° to 80°. In other embodiments, the extended portion (7) can extend at an angle in the range of 50° to 70°. In other embodiments, the extended portion (7) can extend at an angle in the range of 40° to 50°. In other embodiments, the extended portion (7) can extend at an angle in the range of approximately 45° or approximately 75°. The extended portion (7) can extend from the center of the closed end (3) of the finger cover (1). In some embodiments, the grip (6) extends 1 inch from the closed end (3). In other embodiments, the grip (6) extends 0.5, 0.75, 1.25, 1.5, 1.75, or 2 inches, or any range thereof from the closed end (3). Also, the extended portion (7) can be curved such that the curve extends in the same direction as the plurality of protrusions (5). For instance, the extended portion (7) can be curved such that the grip (6) bends toward the center of the arc length (AL). The curvature of the extended portion (7) and the grip (6) can prevent sharp edges or corners that could irritate the walls of the vaginal canal.

The grip can also have an enlarged portion (8) extending from the extended portion (7). The enlarged portion (8) can provide additional removal force by hooking into a retained tampon. Also, the enlarged portion (8) can extend towards the plurality of protrusions (5) so that the plurality of protrusions (5) and the grip (6) can concurrently assist in the removal of the retained tampon. The shape of the enlarged portion (8) is not particularly limited but can include a rounded top portion and a flat bottom portion. The enlarged portion (8) can be centered on the end of the extended portion (7). Also, the enlarged portion (8) can be offset on the end of the extended portion (7) to provide a smooth surface on the grip (6) thereby preventing sharp edges or corners that could irritate the walls of the vaginal canal. The enlarged portion (8) can extend in the same direction as the plurality of protrusions (5). For instance, the enlarged portion (8) can be curved such that the grip (6) bends toward the center of the arc length (AL).

The removal devices disclosed herein can be made from any acceptable material. In certain embodiments, the removal devices are produced through a molding process. In particular, the removal devices can be made from materials that can be molded, such as medical-grade silicone. The removal devices can also be made from materials such as rubber, thermoplastic elastomers including styrenic block copolymers (TPS (TPE-s)), thermoplastic polyolefinelastomers (TPO (TPE-o)), thermoplastic vulcanizates (TPV (TPE-v or TPV)), thermoplastic polyurethanes (TPU (TPU)), thermoplastic copolyester (TPC (TPE-E)), thermoplastic polyamides (TPA (TPE-A)), and not classified thermoplastic elastomers (TPZ), and vinyl. The removal devices can be made from a single material or a mixture of different materials. Since the removal devices may be inserted into the vaginal canal, the material utilized to produce the removal device should be compatible with lubricants that can assist with the insertion into the vaginal canal.

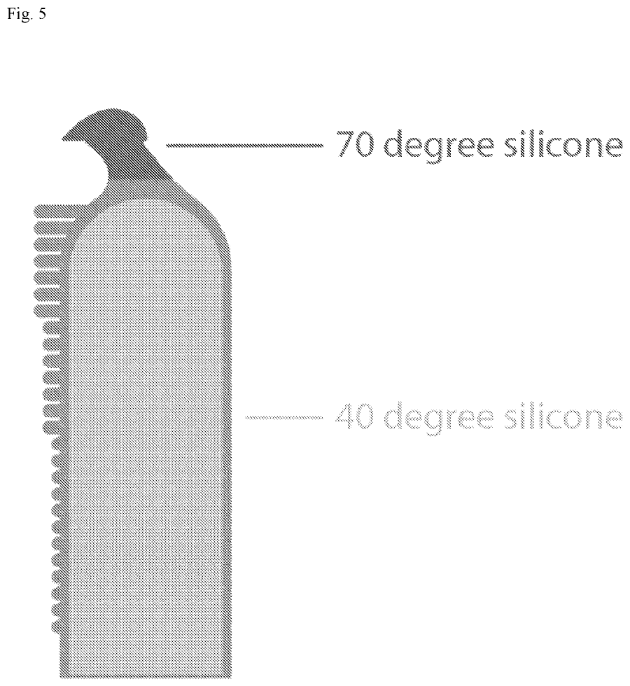

FIG. 5 depicts an embodiment of the removal device made from materials having different Shore hardness values. As seen the embodiment depicted in FIG. 5, at least the grip (6), the extended portion (7), and the enlarged portion (8), can have a higher Shore hardness value than the portion generally made of the finger cover (4). The higher Shore hardness values of at least the grip (6), the extended portion (7), and the enlarged portion (8) allow for the removal devices to secure a retained tampon more easily and assist in the removal thereof. FIG. 5 depicts a Shore hardness value of 70 for the grip (6), the extended portion (7), and the enlarged portion (8), but acceptable Shore hardness values can include values of 40, 50, 60, 65, 70, 75, 80, and 90. For instance, ranges of acceptable Shore hardness values can include 40-90, 60-80, and 65-75. The portion generally made of the finger cover (4) can have the same or a different Shore hardness value as the grip (6), the extended portion (7), and the enlarged portion (8). In general, the Shore hardness value of the portion generally made of the finger cover (4) is less than the grip (6), the extended portion (7), and the enlarged portion (8). The lower Shore hardness values provide greater flexibility to the portion generally made of the finger cover (4), which can improve comfort and wearability for the user. Acceptable Shore hardness values for the portion generally made of the finger cover (4) include values of 20, 30, 35, 40, 45, 50, 60, 65, 70, 75, 80, and 90. For instance, ranges of acceptable Shore hardness values include 20-70, 30-50, and 35-45.

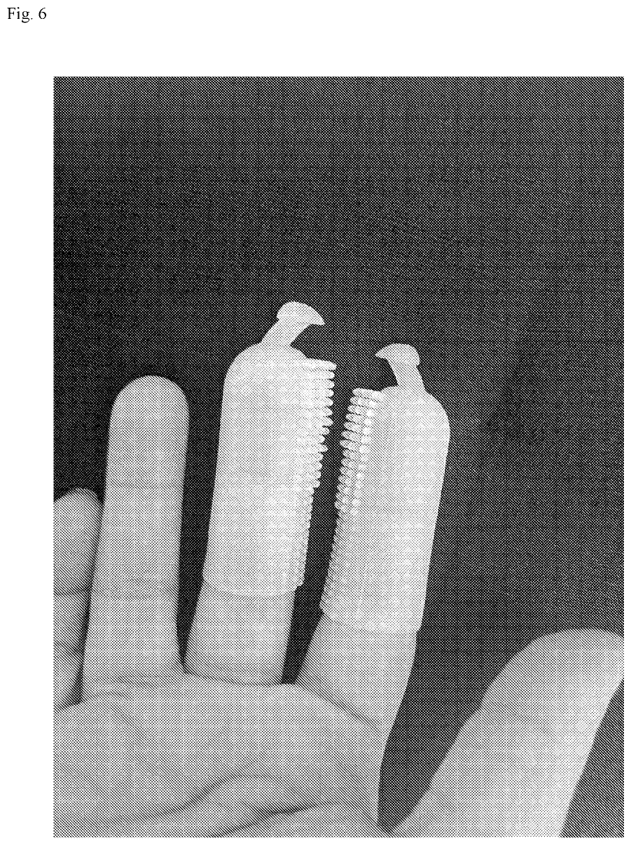

Additionally, the removal devices can be used in a method of removing a retained tampon that involves inserting one or more removal devices into a vaginal cavity having the retained tampon therein, securing the retained tampon with the removal device, and removing the retained tampon with the removal device. The insertion into the vaginal cavity can be assisted with an external lubricant before and/or during the insertion. Further, the user can insert more than one removal device at the same time. For instance, as shown in FIG. 6, the user could place one removal device on their index finger and one removal device on their middle finger and then use the two removal devices together. Alternatively, the user could place one removal device on their index finger and one removal device on their thumb and then use the two removal devices together. As seen in FIG. 6, the removal devices can be turned on the user's fingers such that protrusions on each device face each other, which can assist with the removal of the retained tampon. The removal device can even be connected together at the open end (2). As discussed above, the method can also be utilized to remove objects from other bodily cavities, such as the mouth, ears, anal canal, rectum, etc. The removal devices disclosed herein can even further be used in a non-medical setting, such as being used in a construction setting (e.g., to remove small pieces from a pipe).

### Reference Character List

1—finger cover
2—open end
3—closed end
4—cylindrical portion
5—protrusions
6—grip
7—extended portion
8—enlarged portion
h—cylindrical portion height
d—cylindrical portion external diameter
t—cylindrical portion wall thickness
ph1—protrusions having a first height
ph2—protrusions having a second height
ph3—protrusions having a third height
h1—ph1 surface area height
h2—ph2 surface area height
h3—ph3 surface area height
AL—arc length
C—circumference

In the foregoing description, the removal devices and methods of the present application have been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification, unless the context requires otherwise, the word “comprise” and its variations, such as “comprises” and “comprising,” will be understood to imply the inclusion of a stated item, element or step or group of items, elements or steps, but not the exclusion of any other item, element or step or group of items, elements or steps. Furthermore, the indefinite article “a” or “an” is meant to indicate one or more of the item, element or step modified by the article.

1. A removal device for removing an object from a cavity comprising a finger cover (1),
wherein the finger cover (1) comprises an open end (2), a closed end (3), and a cylindrical portion (4),
wherein the finger cover (1) comprises a plurality of protrusions (5) on at least a portion of an exterior surface of the cylindrical portion (4), and
wherein the finger cover (1) comprises a grip (6) extending from the closed end (3).

2. The removal device of claim 1, wherein the open end (2) is configured to accept a finger of a user.

3. The removal device of claim 1, wherein the closed end (3) has a convex shape.

4. The removal device of claim 1, wherein the cylindrical portion (4) is a right cylinder.

5. The removal device of claim 1, wherein the plurality of protrusions (5) extend radially from the exterior surface of the cylindrical portion (4).

6. The removal device of claim 1, wherein the plurality of protrusions (5) are present on a predefined arc length of the cylindrical portion (4).

7. The removal device of claim 6, wherein the predefined arc length is 20% to 40% of a circumference of the cylindrical portion (4).

8. The removal device of claim 1, wherein the plurality of protrusions (5) are present on the cylindrical portion (4) along a height (h) extending from the open end (2) to the closed end (3).

9. The removal device of claim 1, wherein the plurality of protrusions (5) are evenly spaced on the exterior surface of the cylindrical portion (4).

10. The removal device of claim 1, wherein the plurality of protrusions (5) comprises protrusions having a first height (ph1), protrusions having a second height (ph2), and protrusions having a third height (ph3).

11. The removal device of claim 10, wherein the first height (ph1) is less than the second height (ph2), and wherein the second height (ph2) is less than the third height (ph3).

12. The removal device of claim 10,
wherein the protrusions having a first height (ph1) are closest to the open end (2),
wherein the protrusions having a third height (ph3) are closest to the closed end (3), and
wherein the protrusions having a second height (ph2) are between the protrusions having a first height (ph1) and the protrusions having a third height (ph3).

13. The removal device of claim 1, wherein the grip (6) comprises an extended portion (7) and an enlarged portion (8).

14. The removal device of claim 14, wherein the extended portion (7) curves toward a center of the plurality of protrusions (5).

15. A method of removing a retained tampon comprising
inserting one or more of the removal devices according to claim 1 into a vaginal cavity having the retained tampon therein,
securing the retained tampon with the removal device, and
removing the retained tampon with the removal device.
